(19) United States
(12) Patent Application Publication
(10) Pub. No.: US 2023/0192599 A1
(43) Pub. Date: Jun. 22, 2023

(54) METHOD FOR PREPARING AZO COMPOUND

(71) Applicant: DONGJIN SEMICHEM CO., LTD., Incheon (KR)

(72) Inventors: Min Seung Shin, Siheung-si (KR); Young Gi Kim, Siheung-si (KR); Sok Kyun Choi, Siheung-si (KR); Su Min Son, Siheung-si (KR)

(21) Appl. No.: 18/171,366

(22) Filed: Feb. 19, 2023

Related U.S. Application Data
(63) Continuation of application No. PCT/KR2021/011046, filed on Aug. 19, 2021.

(30) Foreign Application Priority Data
Aug. 19, 2020 (KR) 10-2020-0104216

(51) Int. Cl. C07C 241/02; C07C 281/20

(52) U.S. Cl. CPC C07C 241/02; C07C 281/20

(57) ABSTRACT

A method for preparing an azo compound includes a first step for producing an X_{b} molecule by electrolyzing, in a reaction system, a first solution including a hydrazo compound and at least one type of MₐX_{b}; a second step for oxidizing the hydrazo compound with the generated X_{b} molecule to obtain a second solution including an azo compound, MₐX_{b}, and HX; a third step for discharging the second solution outside the reaction system, and separating therefrom a third solution including MₐX_{b} and HX to obtain a solid azo compound; and a fourth step for introducing the third solution and an additional hydrazo compound equivalent to the hydrazo compound into the reaction system, and electrolyzing a fourth solution including the additional hydrazo compound, MₐX_{b}, and HX to produce an X_{b} molecule.

### CROSS-REFERENCE TO RELATED APPLICATION

This application is a Continuation of International Application No. PCT/KR2021/011046, filed on Aug. 19, 2021, which claims priority to Korean Patent Application No 10-2020-0104216, filed on Aug. 19, 2020. The aforementioned applications are incorporated herein by reference in their entireties.

### TECHNICAL FIELD

The present invention relates to a method for preparing azo compound, and more specifically, to a method for preparing azo compound from a hydrazo compound.

### RELATED ART

An azo compound is a compound having R—N═N—R′ (azo group) (i.e., a structure in which two nitrogen atoms are double bonded), in which R and R′ are each aryl or alkyl. An azo group is a chromophore, and an azo compound including the azo group exhibits colors such as red, orange, and yellow, and thus has high utility value as a dye and is widely used as a colorant of color filters used in display devices (e.g., liquid crystal display panels, electroluminescence, plasma display panels, etc.).

Meanwhile, azodicarbonamide (ADCA), which is a kind of an azo compound, is currently the most commonly used material of foaming agent. The material of foaming agent material is an additive for preparing a porous foam by mixing with a synthetic resin. Azodicarbonamide has a self-extinguishing property and is characterized by non-toxicity, and is used for the purpose of weight lightening, a cushioning property, buoyancy, absorbency, decorativeness, tactility, cost reduction, and dimensional stability of products. Additionally, the foaming of azodicarbonamide is mainly used in polyvinyl chloride (PVC), polyethylene (PE), polypropylene (PP), rubber, an ethylene-vinyl acetate copolymer (EVA), polystyrene (PS), polyurethane (PU), transparent silicone, etc. Additionally, the azodicarbonamide is known as an excellent foaming agent because nitrogen gas is rapidly generated when heated, and decomposition products are non-flammable and non-toxic. Additionally, the azodicarbonamide is also used as a thermostat or bleaching agent for wheat flour (45 ppm or less, US FDA standard).

Meanwhile, azodicarbonamide is usually produced by oxidizing hydrazodicarbon amide (HDCA) with chlorine (Cl₂). In particular, a conventional method of producing azodicarbonamide was to directly supply chlorine to reactants (existing method).

However, according to the existing method, an excess content of chlorine must be used, and since hydrochloric acid (HCl), a strong acid, is produced as a by-product together with azodicarbonamide, there is a problem in that a large content of alkali compound is required for neutralization of the by-product (wastewater). Accordingly, studies have been focused on the development of a method for generating chlorine (Cl₂) by electrolysis. However, this method also had a problem in that it is essential between the positive electrode and negative electrode compartments be separated within a reactor (electrolyzer) through a separator so as to prevent sodium hydroxide (NaOH), a by-product generated in the negative electrode compartment, from decomposing the azodicarbonamide generated in the positive electrode compartment, and a problem in that it requires a large cost for the treatment of the by-product (wastewater), and thus have not been used.

FIG. 1 is a diagram for explaining an electrolysis device and a manufacturing process used for producing an azo compound according to the existing tehcnology.

Referring to FIG. 1, a separator 13 is provided in a container 10, and the container 10 is partititioned into a negative electrode compartment 14 and a positive electrode compartment 15 by a separator 13, the negative electrode 11 is disposed on the negative electrode compartment 14, and the positive electrode 12 is disposed on the positive electrode compartment 15. A stirrer 16 is further provided in the positive electrode compartment 15. A solution 17 containing a hydrazo compound and sodium chloride (NaCl) is put into the container 10, and an azo compound is formed from the hydrazo compound through an electrolysis reaction.

According to the conventional technology of FIG. 1, a method is used where sodium chloride (NaCl) is added to the reactants and chlorine is generated in the reactant through electrolysis and supplied, it is essential that the positive electrode compartment 15 and the negative electrode compartment 14 be separated within a reactor (electrolyzer) [i.e., vessel 10] through the separator 13 so as to prevent sodium hydroxide (NaOH) generated in the negative electrode compartment from decomposing the azodicarbonamide generated in the positive electrode compartment. Since the reaction for producing an azo compound is a slurry reaction, it is essential to stir the reactants for a smooth reaction. The separator 13 is usually formed of a thin membrane, but there is a high likelihood that the separator 13 is damaged by the stirring force of the stirrer 16. Additionally, according to the conventional technology, sodium chloride must be continuously supplied to the reactants so as to continuously produce chlorine that oxidizes the hydrazo compound.

### SUMMARY

The technical object to be achieved in the present invention is to provide a method for producing an azo compound, which, by using a predetermined halogen compound (MₐX_{b}) in producing an azo compound from a hydrazo compound, does not require continuous introduction of a chlorine source, etc., can significantly reduce the burden of treatment of wastewater and by-products, and can realize a high conversion rate and a high yield.

Additionally, the technical object to be achieved in the present invention is to provide a method for producing an azo compound, which does not require the use of a separator even if the electrolysis method is used and can reduce power consumption compared to the conventional technology.

The problems to be solved in the present invention are not limited to those mentioned above, and other problems not mentioned will be clearly understood by those skilled in the art from the following description.

According to embodiments of the present invention for achieving the above objects, there is provided a method for preparing azo compound, which includes: a first step for producing an X_{b} molecule by electrolyzing, in a reaction system, a first solution including a hydrazo compound and at least one type of MₐX_{b}; a second step for oxidizing the hydrazo compound with the generated X_{b} molecule to obtain a second solution including an azo compound, MₐX_{b}, and HX; a third step for discharging the second solution outside the reaction system, and separating therefrom a third solution including MₐX_{b} and HX to obtain a solid azo compound; and a fourth step for introducing the third solution and an additional hydrazo compound equivalent to the hydrazo compound into the reaction system, and electrolyzing a fourth solution including the additional hydrazo compound, MₐX_{b}, and HX to produce an X_{b} molecule, wherein the fourth step, the second step, and the third step are collectively defined as a single cycle, and the cycle is performed repeatedly, and wherein: X is a halogen element; M is at least one selected from hydrogen, Li, Na, K, Mg, Ca, Mn, Fe, Ni, Cu, Ag, Zn, Sn, Zr, and Ti, or at least one selected from a primary ammonium ion, a secondary ammonium ion, and a tertiary ammonium ion; the H is hydrogen; and a and b are each independently any one integer from 1 to 4.

The content of the at least one type of MₐX_{b} initially introduced into the reaction system in the first step may be 1 wt % to 30 wt % based on the total weight of the first solution.

The MₐX_{b} may include at least one of a Cl₂ precursor and a Br₂ precursor.

The content of the Cl₂ precursor may be 3 wt % to 15 wt % based on the total weight of the first solution.

The content of the Br₂ precursor may be 0.05 wt % to 5 wt % based on the total weight of the first solution.

The method for preparing azo compound may satisfy the following Relational Equation (1).

$$0.1 \leq \sqrt{\frac{{α}^{2}}{β}} \leq 9.5 \qquad [ Relational Equation ( 1 ) ]$$

In Relational Equation (1) above, α is the content (wt %) of MₐX_{b} based on the total weight of the first solution, and β is the reaction temperature (° C.) of the method for producing an azo compound.

The method for preparing an azo compound may satisfy the following Relational Equation (1-1).

$$0.33 \leq \sqrt{\frac{{α}^{2}}{β}} \leq 6.35 \qquad [ Relational Equation ( 1 - 1 ) ]$$

In Relational Equation (1-1) above, α is the content (w %) of MₐX_{b} based on the total weight of the first solution, and β is the reaction temperature (° C.) of the method for producing an azo compound.

The concentration of HX can be maintained uniformly in the first to third solutions from the starting time of the first step to the ending time of the third step.

The reaction system may include a solution of any one of the first to fourth steps; a positive electrode immersed in the solution; and a negative electrode immersed in the solution; and the solution around the positive electrode and the negative electrode may be acidic.

The negative electrode may be in direct contact with any one or more of the hydrazo compound and the azo compound.

The hydrazo compound may be hydrazodicarbonamide (HDCA) and the azo compound may be azodicarbonamide (ADCA).

The azo compound may be present in a slurry state before separating the third solution in the third step.

According to another embodiment of the present invention for achieving the above objects, there is provided a method for preparing azo compound, which includes: a first step for producing an X_{b} molecule by electrolyzing, in a reaction system, a first solution including a hydrazo compound and at least one type of MₐX_{b}; a second step for oxidizing the hydrazo compound with the generated X_{b} molecule to obtain a second solution including an azo compound, MₐX_{b}, and HX; and a third step for discharging the second solution outside the reaction system, and separating therefrom a third solution including MₐX_{b} and HX to obtain a solid azo compound; wherein the pH of the first solution to the third solution in the first step to third step reaction systems is uniform, and wherein: X is a halogen element; M is at least one selected from hydrogen, Li, Na, K, Mg, Ca, Mn, Fe, Ni, Cu, Ag, Zn, Sn, Zr, and Ti, or at least one selected from a primary ammonium ion, a secondary ammonium ion, and a tertiary ammonium ion; the H is hydrogen; and a and b are each independently any one integer from 1 to 4.

According to embodiments of the present invention, in a method for producing an azo compound from a hydrazo compound, a method for producing an azo compound with a high conversion rate and a high yield can be realized by using a predetermined halogen compound (MaXb), which does not require continuous introduction of a chlorine source, etc., and can significantly reduce the burden of treatment of wastewater and by-products.

Additionally, according to the embodiments of the present invention, even if the electrolysis method is used, it is unnecessary to use a separator, and it is possible to implement a device for manufacturing an azo compound capable of reducing power consumption compared to the conventional technology. Therefore, the manufacturing process and process management can be facilitated, and manufacturing cost can be reduced, and productivity can be improved.

### BRIEF DESCRIPTION OF THE DRAWINGS

FIG. 1 is a diagram for illustrating an electrolysis device used for producing an azo compound according to the conventional technology and a manufacturing process thereof

FIG. 2 is a flowchart for illustrating a method for preparing an azo compound according to an embodiment of the present invention.

FIGS. 3A, 3B, and 3C are diagrams each showing a reaction system that can be used in the method for preparing an azo compound according to an embodiment of the present invention.

FIG. 4 is a diagram for showing a reaction system that can be used in the method for preparing an azo compound according to another embodiment of the present invention.

FIG. 5 is a diagram for showing a reaction system that can be used in the method for preparing an azo compound according to still another embodiment of the present invention.

### DETAILED DESCRIPTION

Hereinafter, preferred embodiments of the present inventionwill be described in detail with reference to the accompanying drawings.

Examples of the present invention to be described below are provided to more clearly explain the present invention to those skilled in the art, and the scope of the present invention is not limited by the following Examples, and the following Examples can be modified into various other forms.

The terms used herein are used to describe specific embodiments, and not to limit the present invention. As used herein, terms in a singular form may include a plural form unless the context clearly dictates otherwise. Additionally, as used herein, the terms “comprise” and/or “comprising” refer to a referenced shape, step, number, action, member, element, and/or existence of these groups and do not exclude the presence or addition of one or more other shapes, steps, numbers, actions, members, elements, and/or groups thereof. Additionally, as used herein, the term “connection” not only means that certain members are directly connected, but also includes indirectly connected members with other members interposed therebetween.

Additionally, as used herein, when a member is located “on” another member, this includes not only a case in which a member is in contact with another member but also a case in which another member is present between the two members. As used herein, the term “and/or” includes any one and any combination of one or more of those listed items. Additionally, as used herein, terms such as “about”, “substantially”, etc. are used in the meaning of the range or close to the numerical value or degree, in consideration of inherent manufacturing and material tolerances, and are used to prevent the infringer from unfairly using the disclosure, in which the exact or absolute figures are mentioned, provided to help the understanding of the present application.

The size or thickness of the regions or parts shown in the accompanying drawings may be slightly exaggerated for clarity and convenience of description. Like reference numerals refer to like elements throughout the detailed description.

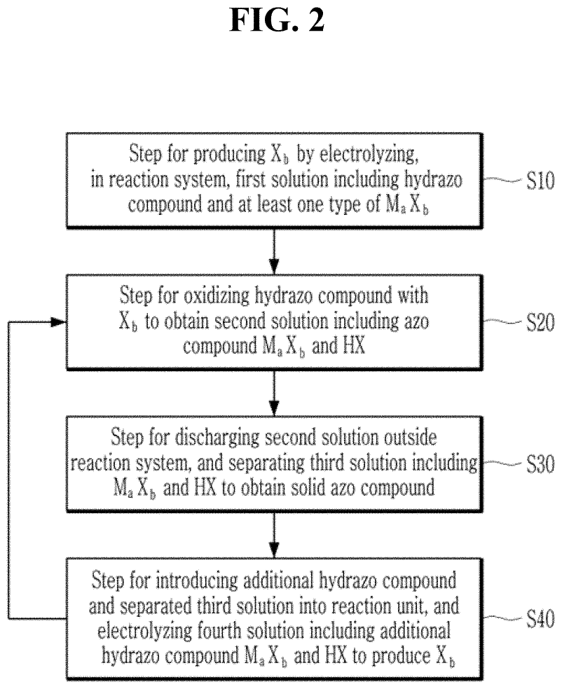

FIG. 2 is a flowchart for illustrating a method for preparing an azo compound according to an embodiment of the present invention.

Referring to FIG. 2, the method for preparing an azo compound according to an embodiment of the present invention may include the following first to fourth steps (S10 to S40).

First step (S10): a step in which a first solution containing a hydrazo compound and at least one kind of MₐX_{b} is introduced into the reaction system, and an electrolysis process is performed on the solution so as to produce X_{b} molecules

Second step (S20): a step in which the hydrazo compound is oxidized with the X_{b} molecules produced so as to obtain a second solution containing an azo compound, MₐX_{b}, and HX

Third step (S30): a step in which the second solution is discharged to the outside of the reaction system, and a third solution containing MₐX_{b} and HX is separated therefrom so as to obtain a solid azo compound

Fourth step (S40): a step in which a hydrazo compound equivalent to the hydrazo compound (an additional hydrazo compound) and the third solution are re-introduceed into the reaction system, and an electrolysis process is performed on a fourth solution containing the hydrazo compound, MₐX_{b}, and HX so as to produce X_{b} molecules

Additionally, in the method for producing an azo compound according to the present embodiment, the fourth step (S40), the second step (S20), and the third step (S30) may be regarded as one cycle and the cycle may be performed repeatedly. That is, after the fourth step (S40), a step corresponding to the second step (S20) and a step corresponding to the third step (S30) may be performed, and after performing the step corresponding to the fourth step (S40) again, a step corresponding to the second step (S20) and a step corresponding to the third step (S30) may be further performed. This process can be performed repeatedly. That is, in the case of a batch reaction, the first step (S10), the second step (S20), the third step (S30), and the fourth step (S40) are sequentially performed. In the case of the batch reaction, when the first to fourth steps (S10 to S40) are performed simultaneously, there may be a problem in terms of reaction stability. Additionally, in the case of a continuous reaction, the reaction may be sequentially performed in the order of the fourth step (S40), the second step (S20), and the third step (S30) and simultaneously. In this case, electrical energy can be continuously applied without interruption.

Here, the X may be a halogen element. For example, the X may include at least one of Cl, Br, and I. The M may be at least one selected from hydrogen, Li, Na, K, Mg, Ca, Mn, Fe, Ni, Cu, Ag, Zn, Sn, Zr, and Ti, or at least one selected from a primary ammonium ion, a secondary ammonium ion, and a tertiary. The ammonium ion may include NH₄ (NH₄). Meanwhile, H represents hydrogen, and a and b may each independently be an integer of any one of 1 to 4.

Hereinafter, each of the above steps (S10 to S40) will be described in more detail.

The first step (S10) may be performed such that a first solution containing a hydrazo compound and at least one kind of MₐX_{b} is introduceed into the reaction system, and an electrolysis process is performed on the first solution to produce X_{b} molecules. In particular, MₐX_{b} may be a halogen compound. In an embodiment, the MₐX_{b} may include any one or more of a Cl₂ precursor and a Br₂ precursor. For example, the MₐX_{b} may include a Cl₂ precursor alone, a Br₂ precursor alone, or include both a Cl₂ precursor and a Br₂ precursor. In particular, the Cl₂ precursor or the Br₂ precursor refers to a material capable of providing Cl₂ or Br₂ through a certain reaction, for example, to a material capable of forming Cl₂ or Br₂ by an electrolysis reaction. The MₐX_{b} may be, for example, HCl. This is the case where M is H and X is Cl in MₐX_{b}. Additionally, the MₐX_{b} may include two or more materials, and may include, for example, HCl and HBr. This is the case where ‘M is H and X is Cl’ and ‘M is H and X is Br’ are combined in MₐX_{b}. In the case of a Br-based compound in which X is Br in MₐX_{b}, it is introduceed as an electrolyte, but it can also serve as a catalyst. In another embodiment, it may include HCl and NaCl. This is the case where ‘M is H and X is Cl’ and ‘M is Na and X is Cl’ are combined in MₐX_{b}. However, the above compounds are exemplary, and other compositions of MₐX_{b} may be used. Meanwhile, since M may be H, MₐX_{b} may be the same as HX.

The first solution may include, for example, water as a solvent. However, the type of solvent is not limited to water and may be variously changed. For example, the solvent may include at least one among water, alcohol, and an organic solvent. In the first solution, the hydrazo compound may exist in a slurry state or in a dissolved state. Even if the hydrazo compound exists in a slurry state, the hydrazo compound can be regarded as a partial constitution of a solution or a constitution included in the solution in a broad sense.

In the first step (S10), X_{b} molecules may be produced by the electrolysis process on MₐX_{b}. The process may be, for example, as shown in Chemical Formula 1 below.

MₐX_{b}→Mₐ+X_{b} [Chemical Formula 1]

During the electrolysis process, Mₐ may be produced in the negative electrode and X_{b} may be produced in the positive electrode. If MₐX_{b} includes HCl, Mₐ may be H₂ (i.e., a hydrogen molecule), and X_{b} may be Cl₂ (i.e., a chlorine molecule). H₂ and Cl₂ may be gases.

If, M in the MₐX_{b} is a metal ion or ammonium ion, Chemical Formula 1 may be changed. In a specific example in Chemical Formula 1, when the MₐX_{b} is 2LiCl, 2Li⁺ and Cl₂ gases may be produced by electrolysis, and when the MₐX_{b} is 2NH₄Cl, 2NH₄⁺ and Cl₂ gases may be produced by electrolysis. Therefore, Chemical Formula 1 above is exemplary, and may vary depending on the material of MₐX_{b} being used. Additionally, when the solvent of the solution is water (H₂O), 2H₂O may be decomposed into H₂ and 2OH⁻ by electrolysis. In this case, the 2Li⁺ may react with 2OH⁻ to become 2LiOH, and the 2NH₄⁺ may react with 2OH⁻ to become 2NH₄OH.

The second step (S20) may be performed such that the hydrazo compound is oxidized with the X_{b} molecules produced to obtain a second solution containing an azo compound, MₐX_{b}, and HX. The reaction in this second step (S20) may be as shown in Chemical Formula 2 below.

a hydrazo compound+X_{b}→an azo compound+2HX [Chemical Formula 2]

In the hydrazo compound, hydrogen (H) may react with X_{b} to form 2HX, and the hydrazo compound may be converted into an azo compound.

The second solution obtained through the second step (S20) may be a solution containing the azo compound, MaXb, and HX. In particular, MₐX_{b} may be a material remaining after consumption of some of the MₐX_{b} introduceed in the first step (S10). For example, when MₐX_{b} includes HCl and HBr in the first step (S10), the HBr may simultaneously serve as a catalyst and may remain without being consumed after participating in the reaction, and thus it may remain in the form of MₐX_{b} in the second step (S20). There is also the possibility that some of the HCl remains. In this regard, it is also possible that the MₐX_{b} in the second step (S20) corresponds to a part of MₐX_{b} introduceed in the first step (S10). In Chemical Formula 2, when the X_{b} molecule is Cl₂, 2HX may be 2HCl. However, the material of 2HX is not limited to 2HCl and may vary. When the Mₐ in MₐX_{b} is H, it may be HX (“first HX”), in which the “first HX” does not refer to HX (“second HX”) produced together with the azo compound but refers to an HX different from the “second HX”. In the second solution of the second step (S20), the azo compound may exist in a slurry state or in a dissolved state. Even if the azo compound exists in a slurry state, the azo compound can be regarded as a part of a solution or a constitution included in the solution in a broad sense.

The third step (S30) may be performed such that the second solution is discharged to the outside of the reaction system, and a third solution containing MₐX_{b} and HX is separated therefrom to obtain a solid azo compound. The second solution obtained in the second step (S20) is discharged to the outside of the reaction system, and then a third solution containing MₐX_{b} and HX is separated from the second solution to obtain a solid azo compound. This may be referred to as a dehydration and drying process to obtain a solid azo compound. Through this, a solid azo compound can be obtained, and simultaneously, a third solution containing MₐX_{b} and HX can be obtained by separation. The solution containing MₐX_{b} and HX separated in this way may be re-introduceed into the reaction system in a subsequent process to be recycled.

That is, when HX is electrolyzed, X_{b} molecules are produced, and simultaneously, the X_{b} molecules oxidize the hydrazo compound to produce an azo compound. Additionally, since HX is produced together with the azo compound as a result of the oxidation reaction, the concentration of HX can be uniformly maintained from the starting time of the second step (S20) to the ending time of the third step (S30). That is, the concentration of the HX in the first to third solutions can be uniformly maintained from the starting time of the first step S10 to the ending time of the third step S30. Therefore, when the solution containing the MₐX_{b} and HX separated in the fourth step (S40) to be described later is re-introduceed after the completion of the third step, the separated solution can be introduceed as-is to proceed with the reaction, and it can be re-introduceed after replenishing only the content of the loss occurred in the separated solution.

The fourth step (S40) may be performed such that a hydrazo compound equivalent to the hydrazo compound (an additional hydrazo compound) and the third solution are introduceed into the reaction system, and an electrolysis process is performed on an additional fourth solution containing the hydrazo compound, MₐX_{b}, and HX to produce X_{b} molecules.

Meanwhile, the term “equivalent” does not mean “the same content” but means “the same compound”.

In the fourth step (S40), X_{b} molecules may be produced by the electrolysis process for the MₐX_{b} and HX. The process may be, for example, as shown in Chemical Formulas 3-1 and 3-2 below.

MₐX_{b}→Mₐ+X_{b} [Chemical Formula 3-1]

2HX→H₂+X₂ [Chemical Formula 3-2]

During the electrolysis process, Mₐ and H₂ may be produced in the negative electrode, and X_{b} and X₂ may be produced in the positive electrode. In particular, X_{b} may include, for example, Cl₂. In the case of Chemical Formula 3-1, as described above in Chemical Formula 1, the chemical formula may be changed depending on the material of MₐX_{b} being used.

The process of producing X_{b} molecules in the fourth step (S40) may correspond to or similar to the process of producing X_{b} molecules in the first step (S10). Accordingly, the fourth step, the second step, and the third step may be regarded as one cycle and the cycle may be performed repeatedly. After the fourth step (S40), a step corresponding to the second step (S20) and a step corresponding to the third step (S30) may be performed, and after performing the step corresponding to the fourth step (S40) again, a step corresponding to the second step (S20) and a step corresponding to the third step (S30) may be further performed. This process may be performed repeatedly.

For example, when HCl is used as a precursor of chlorine (Cl₂), the HCl is electrolyzed to produce chlorine (Cl₂) and simultaneously the chlorine (Cl₂) oxidizes the hydrazo compound. As the hydrazo compound is converted into an azo compound through an oxidation reaction, HCl is produced again. Therefore, the concentration of HCl introduceed at the starting time of the reaction of the first step does not change even though the electrolysis and oxidation reactions are performed repeatedly. That is, the azo compound produced at the ending time of the reaction in the third step and the solution obtained by separating the azo compound can be reused.

Additionally, the separated azo compound may include a trace content of a reaction solution containing HCl, and a water washing process may be performed using a large content of water to remove the trace content of the reaction liquid. The low concentration HCl solution produced through the above process can be concentrated again to a high concentration and reused in the electrolysis reaction of the present invention. The above-described HCl is merely described as an embodiment, and is not limited thereto.

According to this embodiment of the present invention, since the third solution separated in the third step (S30) is recycled and used continuously (repeatedly), there is no need to continuously introduce a new halogen source (e.g., a chlorine source), and the burden of treatment of wastewater and by-products can be significantly reduced.

The content of MₐX_{b} to be initially introduceed into the reaction system in the first step (S10) may be about 1 wt % to about 30 wt % based on the total weight of the first solution. For example, the content of MₐX_{b} to be initially introduceed in the first step (S10) may be about 1 wt % to 20 wt % based on the total weight of the solution containing the hydrazo compound, MₐX_{b}, and HX. When the content of MₐX_{b} initially introduceed into the reaction system in the first step (S10) is less than 1 wt %, the content of the electrolyte is insufficient and the voltage rises, and thus heat is produced, thereby making it difficult to proceed a substantial electrolysis process, whereas when it exceeds 30 wt %, the acid concentration in the solution becomes thick, and thus the production of an azo compound is prevented, and the electrodes where the electrolysis process proceeds may be damaged. The content of MₐX_{b} to be initially introduceed in in the first step (S10) may be determined in consideration of the total weight of the first solution. The content of MₐX_{b} to be initially introduceed in the first step (S10) may be relatively small. The manufacturing process of an azo compound according to the embodiment may be proceeded using a relatively small content of MₐX_{b} only in the initial step (i.e., S10).

When the MₐX_{b} to be introduceed in the first step (S10) includes a Cl₂ precursor, the content of the Cl₂ precursor may be about 3 wt % to 15 wt % based on the total weight of the first solution. When the content of the Cl₂ precursor initially introduceed into the reaction system in the first step (S10) is less than 3 wt %, the voltage is increased due to insufficient content of electrolyte, and subsequently, heat is produced thereby making it difficult to perform an actual electrolysis process, whereas when it exceeds 15 wt %, the acid concentration in the solution becomes thick, and thus the production of an azo compound is prevented, and the electrodes where the electrolysis process proceeds may be damaged.

When the MₐX_{b} to be introduceed in the first step (S10) includes both the Cl₂ precursor and the Br₂ precursor, the content of the Cl₂ precursor may be the same as described above, and the content of the Br₂ precursor may be 0.05 wt % to 5 wt %, preferably 0.1 wt % to 3 wt %, based on the total weight of the first solution. When the content of the Br₂ precursor initially introduceed into the reaction system in the first step (S10) is less than 0.05 wt %, the decomposition temperature of the azo compound being produced is low, and thus the quality may be significantly reduced, whereas when it exceeds 5 wt %, the production yield of the azo compound may be significantly reduced and the amount of electric power per 1 g of the azo compound may be increased.

Additionally, as long as it is a material capable of producing X_{b} molecules by electrolysis in the first step (S10), other materials may also be used even if it is not the above-described MₐX_{b} material.

Meanwhile, the X (a halogen element) in the HX mentioned in the second step (S20), the third step (S30), and the fourth step (S40) may be, for example, at least one of Cl, Br, and I. In other words, the HX may include, for example, at least one selected from the group consisting of HCl, HBr, and HI.

The manufacturing method of the azo compound according to this embodiment may satisfy the following Relational Equation (1), preferably, the following relational equation (1-1).

$$0.1 \leq \sqrt{\frac{{α}^{2}}{β}} \leq 9.5 \qquad [ Relational Equation ( 1 ) ]$$

$$0.33 \leq \sqrt{\frac{{α}^{2}}{β}} \leq 6.35 \qquad [ Relational Equation ( 1 - 1 ) ]$$

In Relational Equation (1-1) above, α is the content (wt %) of MₐX_{b} based on the total weight of the first solution, and β is the reaction temperature (° C.) of the method for preparing an azo compound.

When the numerical value according to the above Relational Equation (1) is less than 0.1 or exceeds 9.5, the electric power consumption per 1 g of the azo compound prepared according to the method for preparing an azo compound according to the present embodiment may be significantly increased, and the quality of the azo compound may be deteriorated by significantly lowering or significantly increasing the decomposition temperature of the azo compound.

The reaction system used in an embodiment of the present invention may include a solution containing the MₐX_{b}, a positive electrode immersed in the solution, and a negative electrode immersed in the solution. Here, the solution may correspond to the solution in any one of the first to fourth steps (S10 to S40). Accordingly, the solution may further include at least one of a hydrazo compound, HX, an azo compound, and a solvent.

In an embodiment of the present invention, the solution may further include an additive if necessary. The additive may be at least one selected from the group consisting of organic acids or inorganic acids, and is not limited as long as it is a material capable of serving as an electrolyte by being dissolved in a solution.

The positive electrode and the negative electrode may be electrodes for the electrolysis reaction in the first step (S10) and the fourth step (S40). For example, the positive electrode may include at least one among titanium (Ti) and alloys thereof, Hastelloy, platinum (Pt) and alloys thereof, stainless steel (e.g., SUS), iridium (Ir) and alloys thereof, an iridium (Ir)-coated metal, ruthenium (Ru) or oxides thereof, graphite, and carbon lead. The negative electrode may include at least one among stainless steel (e.g., SUS), titanium (Ti) and alloys thereof, and aluminum (Al) and alloys thereof. However, the materials of the positive electrode and the negative electrode mentioned above are exemplary, and the present application is not limited thereto. As the material of the positive electrode, an electrode where a noble metal (e.g., gold, silver, platinum, ruthenium, etc.) is coated on a noble metal substrate, a minor metal substrate (e.g., titanium, chromium, nickel, manganese, etc.), and a non-noble metal substrate (e.g., titanium, stainless steel, iron, Hastelloy, etc.); an electrode where a noble metal is coated on a non-metal substrate (e.g., olefin resin, engineering resin, a carbon-based substrate, etc.); a composite electrode coated with platinum and a metal oxide (e.g., iridium oxide or ruthenium oxide); the coated electrode as described above by a minor metal; etc. may be used. The material of the negative electrode is not particularly limited, and all of the materials exemplified as the material of the positive electrode, general-purpose metals (e.g., iron, copper, and aluminum), stainless steel, Hastelloy, various alloys, and a composite electrode provided with the same may be used. The materials of the positive electrode and the material of the negative electrode are not limited as long as they are an electrode material that do not cause corrosion even in an acidic solution.

The solution around the positive electrode and negative electrode may be “acidic”. The pH of the solution in the reaction system may be uniform or substantially uniform. In the conventional technology, the positive electrode compartment and the negative electrode compartment are separated, and the positive electrode compartment shows the acidity of about pH 1 to pH 4, and the negative electrode compartment shows alkalinity of about pH 11 to pH 14. In contrast, according to an embodiment of the present invention, the pH of the solution in the reaction unit may exhibit a uniform (substantially uniform) acidity as a whole. As the pH of the solution in the reaction system becomes low, the yield of the azo compound produced may increase and the quality of the azo compound may be excellent. The pH may represent an acidity of about pH 1 to pH 4, specifically, an acidity of about pH 1 to pH 2.

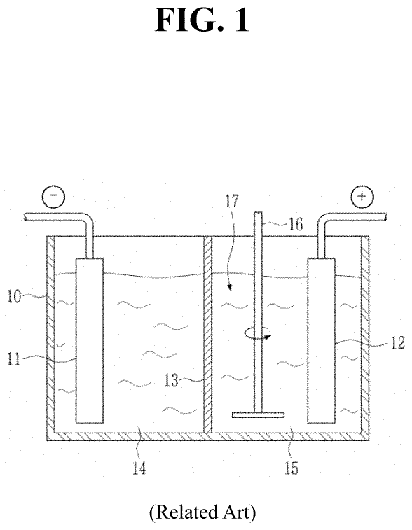

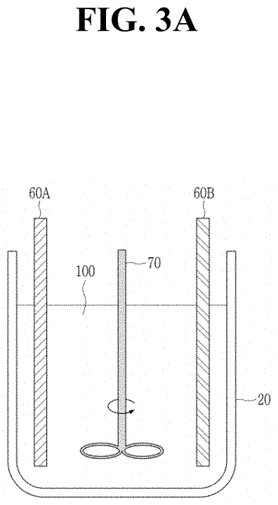

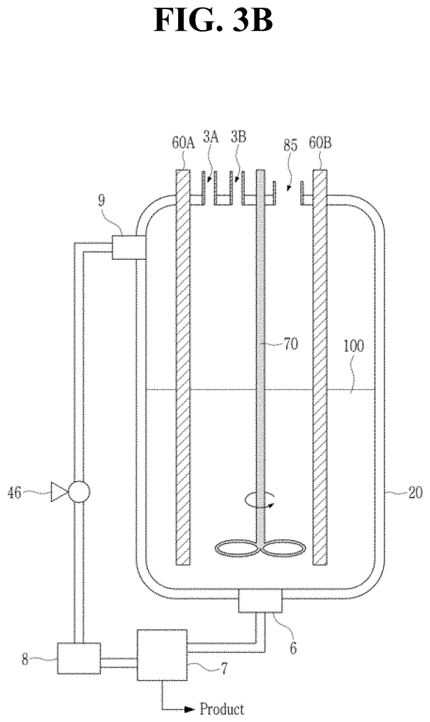

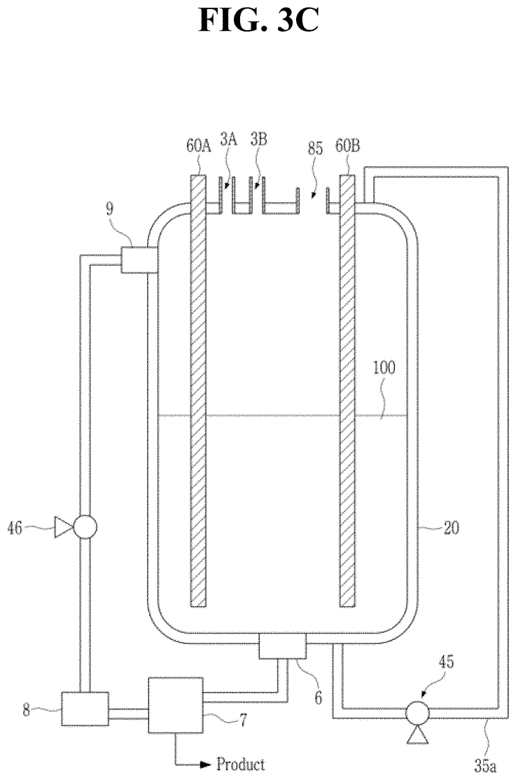

Additionally, in an embodiment of the present invention, the negative electrode may be in direct contact with any one or more of the hydrazo compound and the azo compound. In the conventional device as shown in FIG. 1, in order to prevent decomposition of azodicarbonamide produced in the positive electrode compartment 15, the positive electrode compartment 15 and the negative electrode compartment 14 in the reactor (electrolyzer) (i.e., the vessel 10) must be essentially separated through the separator 13. However, in an embodiment of the present invention, a separator may not be used, and thus, the negative electrode may be in direct contact with any one or more of the hydrazo compound and the azo compound, and the stirring speed can be increased. In this case, since the separator is not used, there are effects in that the manufacturing process and process management can become easier, and there is also an economic advantage in that the separator replacement cost does not occur caused by the breakage of the separator. The above-described reaction system will be described in more detail later with reference to FIGS. 3A to 5.

In the first step (S10) or the fourth step (S40), electrical energy is applied to the reaction unit for the electrolysis, where the electric power applied to the reaction system may be, for example, about 1 W to about 10 W per 1 g of the azo compound. Specifically, the electric power may be about 1.5 W to about 5 W. In this case, for the completion of an electrolysis reaction, for example, it may take about 4 to 6 hours. In a specific embodiment, when a current of about 10 A is applied per 100 g of the hydrazo compound, it may take about 4 to 6 hours. Additionally, electric energy is applied to the reaction system for the electrolysis in the first step (S10) or the fourth step (S40), and the voltage applied to the reaction system may be, for example, about 1 V to 13 V, and specifically, it may be about 2 V to 12 V. The range of the electric power and voltage may be relatively lower than the electric power and voltage used in the device according to the conventional technology described with reference to FIG. 1. Therefore, according to the embodiments of the present invention, it is possible to reduce the electric power consumption and reduce the manufacturing cost compared to the conventional technology.

In the first step (S10), the hydrazo compound may be introduceed, for example, in a slurry type. Additionally, before separating the solution containing MₐX_{b} and HX in the third step (S30), the azo compound may exist, for example, in a slurry state. In this case, there is an advantage in that the hydrazo compound can be more easily converted into the azo compound, and the obtained (synthesized) azo compound can be dehydrated/dried in a relatively simple manner without a complicated process. However, in some cases, the hydrazo compound and/or the azo compound may be in a state dissolved in a solution rather than in a slurry state.

In the method for producing an azo compound according to the embodiments of the present invention described above with reference to FIG. 2, the hydrazo compound may be, for example, hydrazodicarbonamide (HDCA), and the azo compound may be, for example, azodicarbonamide (ADCA). However, these are examples, and the specific materials of the hydrazo compound and the specific material of the azo compound may vary.

Additionally, the method for preparing an azo compound according to an embodiment of the present invention may be performed at a temperature in the range of 10° C. to 80° C., preferably at a temperature in the range of 10° C. to 45° C. In the azo compound manufacturing method, when the temperature is less than 10° C., the reaction rate may be slow or the reaction may not proceed, whereas when the temperature exceeds 80° C., there may be problems in that the azo compound may be decomposed by heat to thereby decrease the yield or deteriorate the quality, and in that the amount of electric power required per weight of the azo compound to be produced may be significantly increased.

Additionally, the method for preparing an azo compound according to an embodiment of the present invention can achieve a yield close to 100%. For example, a high yield of about 90 to 96% can be achieved. Additionally, the method for preparing an azo compound according to this embodiment can be performed in one reaction system from electrolysis to the synthesis of an azo compound.

The method for producing an azo compound according to another embodiment of the present invention may include the following first to third steps (S10 to S30).

First step (S10): a step in which a first solution containing a hydrazo compound and at least one kind of MₐX_{b} is introduceed into the reaction system, and an electrolysis process is performed on the solution so as to produce X_{b} molecules

Second step (S20): a step in which the hydrazo compound is oxidized with the X_{b} molecules produced so as to obtain a second solution containing an azo compound, MₐX_{b}, and HX

Third step (S30): a step in which the second solution is discharged to the outside of the reaction system, and a third solution containing MₐX_{b} and HX is separated therefrom so as to obtain a solid azo compound

The solution around the positive electrode and negative electrode may be “acidic”. The pH of the solution in the reaction system may be uniform or substantially uniform. In the conventional technology, the positive electrode compartment and the negative electrode compartment are separated, and the positive electrode compartment shows the acidity of about pH 1 to pH 4, and the negative electrode compartment shows alkalinity of about pH 11 to pH 14. In contrast, according to an embodiment of the present invention, the pH of the solution in the reaction system may exhibit a uniform (substantially uniform) acidity as a whole. As the pH of the solution in the reaction unit becomes low, the yield of the azo compound produced may increase and the quality of the azo compound may be excellent. The pH may represent an acidity of about pH 1 to pH 4, specifically, an acidity of about pH 1 to pH 2.

Here, the X may be a halogen element. For example, the X may include at least one of Cl, Br, and I. The M may be at least one selected from hydrogen, Li, Na, K, Mg, Ca, Mn, Fe,

Ni, Cu, Ag, Zn, Sn, Zr, and Ti, or at least one selected from a primary ammonium ion, a secondary ammonium ion, and a tertiary. The ammonium ion may include NH₄ (NH₄⁺). Meanwhile, H represents hydrogen, and a and b may each independently be an integer of any one of 1 to 4.

For the specific contents of another embodiment of the present invention, the contents described in the above one embodiment may be equally applied.

FIGS. 3A to 3C are diagrams each showing a reaction system (i.e., a device for preparing an azo compound) that can be used in the method for preparing an azo compound according to an embodiment of the present invention. The reaction system of FIGS. 3A and 3B may be embodiments of the reaction system described with reference to FIG. 2.

Referring to FIGS. 3A to 3C, the reaction system that can be used in the method for preparing an azo compound according to an embodiment of the present invention may include a reaction vessel (i.e., a reaction vessel) 20. The solution 100 for preparing an azo compound according to the embodiment can be contained in the reaction tank 20. In particular, the solution 100 may correspond to the solution in any one of the first to fourth steps (S10 to S40) described in FIG. 2. Accordingly, the solution 100 may be a solution containing the above-described MₐX_{b}, and may further include at least one of the above-described hydrazo compound, HX, azo compound, and solvent.

The reaction system may include a negative electrode 60A and a positive electrode 60B immersed in the solution 100. The negative electrode 60A and the positive electrode 60B are for the electrolysis reaction of the solution 100, and at least a portion of them may be immersed in the solution 100. The electrolysis reaction may correspond to the electrolysis in the first step (S10) and the fourth step (S40) of FIG. 2. For example, the positive electrode 60B may include at least one among titanium (Ti) and alloys thereof, Hastelloy, platinum (Pt) and alloys thereof, stainless steel (e.g., SUS), iridium (Ir) and alloys thereof, iridium (Ir)-coated metals, rubidium (Ru) or oxides thereof, graphite, and carbon lead. The negative electrode 60A may include at least one among stainless steel (e.g., SUS), titanium (Ti) and alloys thereof, and aluminum (Al) and alloys thereof. However, the materials of the positive electrode 60B and the negative electrode 60A listed above are exemplary, and the present invention is not limited thereto. As the material of the positive electrode 60B, an electrode where a noble metal (e.g., gold, silver, platinum, ruthenium, etc.) is coated on a noble metal substrate, a minor metal substrate (e.g., titanium, chromium, nickel, manganese, etc.), and a non-noble metal substrate (e.g., titanium, stainless steel, iron, Hastelloy, etc.); an electrode where a noble metal is coated on a non-metal substrate (e.g., olefin resin, engineering resin, a carbon-based substrate, etc.); a composite electrode coated with platinum and a metal oxide (e.g., iridium oxide or ruthenium oxide); the coated electrode as described above by a minor metal; etc. may be used. The materials for the positive electrode and the material of the negative electrode are not limited as long as they are electrode materials that do not cause corrosion even in an acidic solution.

As the shape of the negative electrode 60A or the positive electrode 60B, a plate material, a punched metal with holes, mesh, porous metal, fiber shape, etc. may be used. The process efficiency can be further improved by variously modifying the shape of the negative electrode 60A or the positive electrode 60B to expand the reaction area. However, the shapes of the negative electrode 60A and the positive electrode 60B are not limited to those described above and other various shapes/structures may be used.

The negative electrode 60A and the positive electrode 60B may be formed as a pair, or may be formed of a plurality of pairs of two or more pairs. For the efficiency of the reaction, it may be more advantageous that the distance between the negative electrode 60A and the positive electrode 60B be close. In an embodiment of the present invention, a separator may not be provided between the negative electrode 60A and the positive electrode 60B. Additionally, the method for connecting the negative electrode 60A and the positive electrode 60B may include a series connection, a parallel connection, or a mixed connection of a series connection and a parallel connection, etc., but is not limited thereto.

Meanwhile, the reaction tank 20 of the reaction system that can be used in the method for producing an azo compound according to an embodiment of the present invention may have an open structure with an open top as shown in FIG. 3A, or a closed structure as shown in FIG. 3B. it may be When the reaction tank 20 has a closed structure as shown in FIG. 3B, it may further include a discharge unit 6 and a gas treatment unit 85 for discharging reactants/products. The gas treatment unit 85 may be provided at an upper end of the reaction tank 20. Various types of gas (e.g., ammonia (NH₃) gas, nitrogen (N₂) gas, hydrogen (H₂) gas, chlorine (Cl₂) gas, bromine (Br₂) gas, etc.) generated in the process of performing the method for producing an azo compound according to an embodiment of the present invention can be captured and used in a variety of ways.

Additionally, as shown in FIG. 3B, in a configuration for recycling the reactants (a hydrazo compound and a solution containing MₐX_{b} and HX), a dehydration unit (product filter) 7, a dehydration mother liquor storage tank 8, a reaction solution transfer pump 46, and a recycling unit 9 may be further included.

Referring to FIG. 3B, when the reactants/composites are discharged through the discharge unit 6, the azo compound may be separated through the dehydration unit 7. In particular, the dehydration unit 7 may fulfill centrifugal separation, reduced pressure filtration, etc. that are generally used. The solution (a solution containing MₐX_{b} and HX) remaining after separation of the azo compound through the dehydration unit 7 may pass through the dehydration mother liquid storage tank 8, and through the dehydration mother liquid storage tank 8, the separated solution is re-introduceed into the reaction system through the recycling unit 9 installed in the reaction system by the reaction liquid transfer pump 46. In particular, the dehydration mother liquid storage tank 8 and the reaction liquid transfer pump 46 may be disposed in the order shown in FIG. 3B or may be disposed in a reversed order of positions.

A filtration unit may be included as needed. Impurities that may be included in the reactants can be filtered through the filtration unit.

Additionally, the reaction system may further include a stirrer 70 for stirring a solution 100 as shown in FIGS. 3A and 3B. When the solution 100 is properly stirred using the stirrer 70, the reaction may proceed more smoothly, and the efficiency may be increased. The form of the stirrer 70 shown here is merely exemplary, and various kinds of stirrers (a wing type, a magnetic bar type, etc.) may be used. The appropriate stirring speed (rpm) may vary depending on the type of stirrer 70 selected.

It is also possible not to use the stirrer 70, and as shown in FIG. 3C, when the reaction system does not include a stirrer, the solution can be stirred using an external power (i.e., the pump 45). In particular, the pump 45 is connected to the reaction tank 20 through the connecting pipe 35a. Additionally, through the connecting pipe 35a as a passage, the solution moves from a lower part to an upper part of the reaction tank 20, and as a result, the effect of stirring the solution in the reaction tank 20 can be achieved.

The process for preparing an azo compound according to the embodiment of the present invention described with reference to FIG. 2 may be performed using the reaction system (i.e., a device for preparing an azo compound) as shown in FIGS. 3A to 3C. Accordingly, all of the specific manufacturing processes described with reference to FIG. 2 may be applied to the reaction system of FIGS. 3A to 3C.

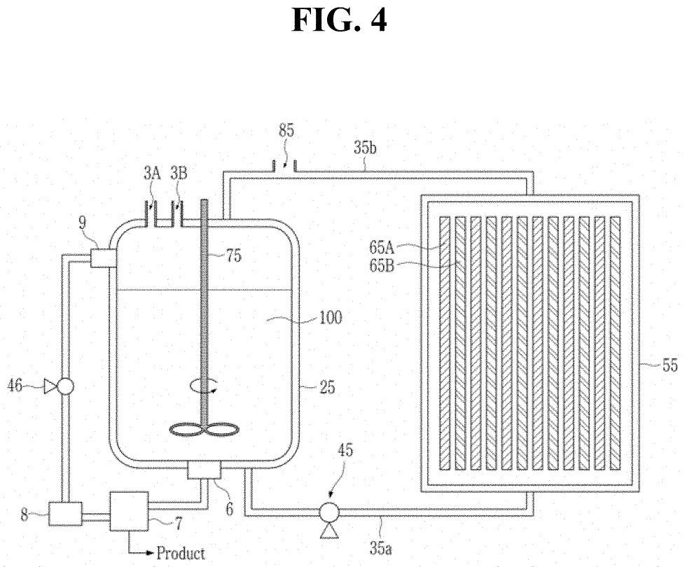

FIG. 4 is a diagram showing a reaction system (i.e., a device for preparing an azo compound) that can be used in a method for preparing an azo compound according to another embodiment of the present invention. The reaction system of FIG. 4 may be an embodiment of the reaction system described with reference to FIG. 2.

Referring to FIG. 4, the reaction system that can be used in the method for preparing an azo compound according to an embodiment of the present invention may include a reaction tank (i.e., a reaction vessel) 25. The solution 100 for preparing an azo compound according to the embodiment may be immersed in the reaction tank 25. In particular, the solution 100 may correspond to the solution in any one step of the first to fourth steps (S10 to S40) described with reference to FIG. 2. Accordingly, the solution 100 may be a solution containing the aforementioned MaXb, and may further include at least one of the hydrazo compound, HX, azo compound, and solvent. The reaction tank 25 may be provided with a reaction solution introduction unit 3A for introducing a solution containing MₐX_{b} and HX, a hydrazo compound introduction unit 3B for input of a hydrazo compound, and a discharge unit 6 for discharging reactants/products The hydrazo compound may be introduced in the form of a slurry. The positions, shapes, structures, etc. of the introduction units 3A and 3B and the discharge part 6 are exemplary and may be variously changed.

The reaction systemof this embodiment may further include an electrode chamber (i.e., an electrode tank) 55 spaced apart from the reaction tank 25. At least one negative electrode 65A and at least one positive electrode 65B may be provided in the electrode tank 55. One or more pairs of the negative electrode 65A and the positive electrode 65B may be disposed in the electrode tank 55. The negative electrode 65A and the positive electrode 65B are for the electrolysis reaction of the solution 100, and the electrolysis reaction can correspond to the electrolysis in the first step (S10) and the fourth step (S40) with reference to FIG. 2. The specific materials of the negative electrode 65A and the positive electrode 65B may be the same as those described with reference to FIGS. 3A to 3C.

The reaction system of this embodiment may further include a gas treatment unit 85. The gas treatment unit 85 may be provided at an upper end of the reaction tank 25 and the electrode chamber 55. Various types of gas (e.g., ammonia (NH₃) gas, nitrogen (N₂) gas, hydrogen (H₂) gas, chlorine (Cl₂) gas, bromine (Br₂) gas, etc.) generated in the process of performing the method for producing an azo compound according to an embodiment of the present invention can be captured and used in a variety of ways.

The reaction system of this embodiment may further include a connection structure which connects the reaction tank 25 and the electrode tank 55. The connection structure may include, for example, a first connecting pipe 35a and a second connecting pipe 35b. The first connecting pipe 35a may be configured to connect a first end of the reaction tank 25 and a first end of the electrode tank 55, and the second connecting pipe 35b may be configured to connect a second end of the reaction tank 25 and a second end of the electrode tank 55. A pump 45 may be installed in the first connecting pipe 35a. The pump 45 may be a kind of circulation pump.

The solution 100 can be circulated within the reaction system by the operation of the pump 45. In other words, by the operation of the pump 45, the solution 100 moves from the reaction tank 25 to the electrode tank 55 through the first connecting pipe 35a, and is then introduceed from the electrode tank 55 back into the reaction tank 25 through the second connecting pipe 35b.

Additionally, the reaction unit of this embodiment may further include a stirrer 75 for stirring the solution 100 in the reaction tank 25. Various types of the stirrer 75 may be used, and an appropriate stirring speed may vary depending on the type of the stirrer 75.

Additionally, as shown in FIG. 3B, in a configuration for recycling the reactants (a hydrazo compound and a solution containing MₐX_{b} and HX), a dehydration unit (product filter) 7, a dehydration mother liquor storage tank 8, a reaction solution transfer pump 46, and a recycling unit 9 may be further included.

The process for preparing an azo compound according to the embodiment of the present invention described with reference to FIG. 2 may be performed using the reaction system (i.e., a device for preparing an azo compound) as shown in FIG. 4. Accordingly, all of the specific manufacturing processes described with reference to FIG. 2 may be applied to the reaction system of FIG. 4.

In the case of the reaction system described in FIG. 4, since the solution 100 may be circulated by the pump 45, the stirrer 75 may not be provided in the reaction tank 25. That is, since an effect similar to stirring can be obtained by circulation of the solution 100 by the pump 45, a separate stirrer 75 may not be provided.

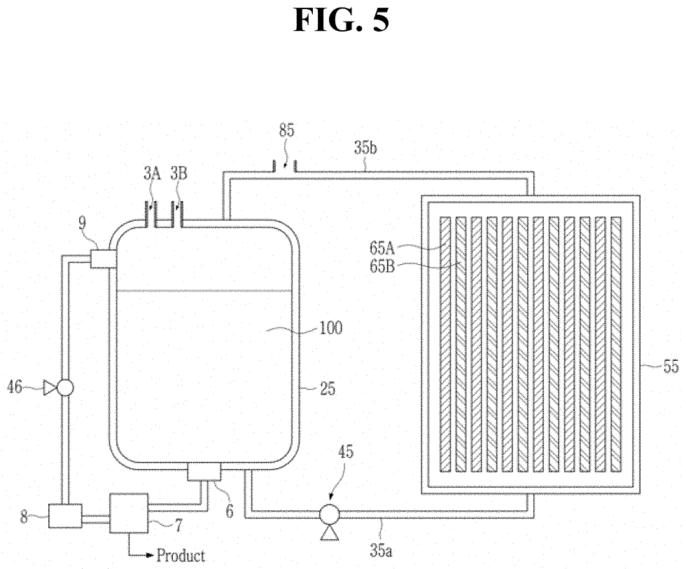

The reaction system excluding the stirrer 75 in FIG. 4 may be the same as shown in FIG. 5. The reaction system of FIG. 5 may be the same as the reaction system of FIG. 4 except that it does not include a stirrer.

### EXAMPLES

Hereinafter, an azo compound prepared by the method for preparing an azo compound according to an embodiment of the present invention will be described in detail with reference to Examples and Comparative Examples. Additionally, the Examples shown below are an embodiment to help understanding of the present invention, and the scope of the present invention is not limited.

### Method for Producing an Azo Compound

### Example 1

A 500 mL beaker, electrodes, hydrazodicarbonamide (HDCA), distilled water, and MₐX_{b} were prepared for an electrolysis reaction. HDCA, distilled water, and MₐX_{b} were each weighed and introduceed into the 500 mL beaker according to the contents shown in Table 1 below. Thereafter, the introduceed materials were sufficiently stirred using a stirrer.

Double boiling was performed in a reactor suitable for the reaction temperature using water, and the mixture was stirred at a temperature corresponding to the experimental conditions through a temperature control unit for 30 minutes to 1 hour to maintain a uniform temperature.

The negative electrode and the positive electrode in the reactor were were installed such that they are in the form to face each other while maintaining a distance of 1 mm to 5 mm, and the facing electrodes are immersed in the solution. In particular, the electrodes were prepar in a structure not to contact with each other and a gap was maintained in a constant state.

Thereafter, the mixture was carefully stirred using a stirrer so that the stirring could be proceeded in a state where no impact was applied to the electrodes. In particular, a turbine-type stirring blade with a diameter of 3 cm was used, and the RPM was maintained at 300 RPM.

The negative electrode and the positive electrode were connected to each electrode immersed in the solution in the reactor using a power supply, and a constant current was supplied to flow.

Upon confirming that all of the reactants were converted into a product, the supply of electricity was stopped and the product was separated using a reduced pressure filter.

### Examples 2 to 30 and Comparative Example 1

The preparation was performed in the same manner as in Example 1, but an azo compound was prepared as in Table 1 below.

Regarding the quality of the obtained azodicarbonamide (ADCA) described in Table 1 below, the meanings of the following indications are as follows.

[symbol] : It can be used as a high-quality product due to uniform particle shape and particle size.

∘: Although the particle shape and particle size are uniform, it is difficult to be used as a high-quality product because it includes materials with some different particle sizes, and it can be used as a general product.

Δ: The particle shape and particle size are not uniform, and thus it can be used as a product only after separation.

X: The particle shape is poor and the particle size distribution is wide, and thus it cannot be used as a product.

TABLE 1

| | | | | | | | | | | | Amount of | |
|---|---|---|---|---|---|---|---|---|---|---|---|---|
| | | | | | | | | | | Content | Electric | |
| | | MₐX_{b} | Solvent | HDCA | | | Temper- | | | of ADCA | Power per 1 | |
| | | Content | Content | Content | Current | Time | ature | | Yield | Acquired | g of ADCA | ADCA |
| | MₐX_{b} | (wt %) | (wt %) | (wt %) | (A) | (h) | (° C.) | pH | (%) | (g) | (W/g) | Quality |
| Example 1 | HCl | 6 | 69 | 25 | 10 | 1.25 | 40 | 1 | 94 | 23.1 | 1.56 | ⊚ |
| Example 2 | NaCl | 6 | 69 | 25 | 10 | 1.918 | 40 | 4 | 82 | 20.2 | 8.71 | ⊚ |
| Example 3 | KCl | 6 | 69 | 25 | 10 | 2 | 40 | 4 | 85 | 20.9 | 9.68 | ⊚ |
| Example 4 | MgCl₂ | 6 | 69 | 25 | 10 | 1.375 | 40 | 3 | 91 | 22.6 | 6.25 | ⊚ |
| Example 5 | HCl | 0.5 | 74.5 | 25 | 10 | — | 40 | 2 | — | — | — | X |
| Example 6 | HCl | 1 | 74 | 25 | 10 | 1.85 | 40 | 1 | 93 | 22.9 | 4.68 | ◯ |
| Example 7 | HCl | 3 | 72 | 25 | 10 | 1.5 | 40 | 1 | 93 | 22.9 | 1.9 | ⊚ |
| Example 8 | HCl | 15 | 60 | 25 | 10 | 1.333 | 40 | 1 | 92 | 22.5 | 1.59 | ⊚ |
| Example 9 | HCl | 17 | 58 | 25 | 10 | 1.333 | 40 | 1 | 88 | 21.625 | 1.66 | ◯ |
| Example 10 | HCl | 30 | 45 | 25 | 10 | 1.3 | 40 | 1 | 80 | 19.7 | 1.50 | ◯ |
| Example 11 | HCl | 32 | 43 | 25 | 10 | 1.4 | 40 | 1 | 70 | 17.2 | 2.01 | Δ |
| Example 12 | NaCl | 0.5 | 74.5 | 25 | 10 | — | 40 | — | — | — | — | X |
| Example 13 | NaCl | 1 | 74 | 25 | 10 | 2.1 | 40 | 3 | 80 | 19.7 | 2.88 | ◯ |
| Example 14 | NaCl | 3 | 72 | 25 | 10 | 2.003 | 40 | 4 | 82 | 20.2 | 2.68 | ◯ |
| Example 15 | NaCl | 15 | 60 | 25 | 10 | 2.1 | 40 | 4 | 75 | 18.4 | 3.07 | ◯ |
| Example 16 | NaCl | 17 | 58 | 25 | 10 | 3.125 | 40 | 5 | 50 | 12.3 | 6.86 | Δ |
| Example 17 | NaCl | 32 | 43 | 25 | 10 | 3.27 | 40 | 6 | 20 | 4.9 | 17.95 | X |
| Example 18 | KCl | 0.5 | 74.5 | 25 | 10 | — | 40 | — | — | — | — | X |
| Example 19 | KCl | 1 | 74 | 25 | 10 | 2.15 | 40 | 4 | 85 | 20.9 | 9.68 | ◯ |
| Example 20 | KCl | 3 | 72 | 25 | 10 | 2.075 | 40 | 4 | 85 | 20.9 | 2.68 | ◯ |
| Example 21 | KCl | 15 | 60 | 25 | 10 | 2.15 | 40 | 4 | 75 | 18.4 | 3.14 | ◯ |
| Example 22 | KCl | 17 | 58 | 25 | 10 | 3.075 | 40 | 5 | 52 | 12.8 | 6.49 | Δ |
| Example 23 | KCl | 32 | 43 | 25 | 10 | 3.75 | 40 | 6 | 22 | 5.4 | 18.72 | X |
| Example 24 | HCl | 6 | 69 | 25 | 5 | 2.668 | 40 | 1 | 92 | 22.6 | 1.64 | ⊚ |
| Example 25 | HCl | 3 | 72 | 25 | 5 | 2.918 | 40 | 1 | 90 | 22.1 | 1.4 | ⊚ |
| Example 26 | HCl | 6 | 81.5 | 12.5 | 10 | 0.65 | 40 | 1 | 94 | 11.6 | 1.63 | ⊚ |
| Example 27 | HCl | 6 | 54 | 40 | 10 | 2.05 | 40 | 1 | 94 | 37.0 | 1.61 | ⊚ |
| Example 28 | HCl | 6 | | recycle | 25 | 10 | 1.25 | 40 | 1 | 94 | 46.2 | |
| | | | | once | | | | | | | | |
| Example 29 | HCl | 6 | | recycle | 25 | 10 | 1.25 | 40 | 1 | 94.5 | 139.4 | |
| | | | | 5 times | | | | | | | | |
| Example 30 | HCl | 6 | | recycle | 25 | 10 | 1.25 | 40 | 1 | 94.5 | 255.5 | |
| | | | | 10 times | | | | | | | | |
| Comparative | HCl | 6 | 69 | 25 | 10 | — | 40 | 1 | 0 | 0 | — | X |
| Example 1 | | | | (urea) | | | | | | | | |

In Table 1, the total mass of the solution containing MₐX_{b}, HDCA, and the solvent is based on 100 g, and the time is based on 25 g of HDCA.

Referring to Table 1, in Examples 1 to 4, in which the type of material for supplying chlorine (Cl₂) (i.e., MₐX_{b}) was changed, it was confirmed that although HCl, NaCl, KCl, MgCl₂, HBr, etc. are all possible, ADCA of the best quality was obtained in the case of HCl.

In Examples 5 to 23, the content of MₐX_{b} was changed, and it was confirmed that when the content was less than 1 wt %, ADCA was not produced, whereas when it exceeded 30 wt %, ADCA of low quality was obtained with a yield of 70% or less.

In Examples 24 and 25, the amount of current was changed to be lower than those of other examples, and it was confirmed that although the reaction time was slightly longer, the quality of the ADCA was excellent.

In Examples 26 and 27, the content of HDCA was changed to be lower or higher than those of other examples, and it was confirmed that the quality of ADCA was all excellent regardless of the change in the content of HDCA.

Examples 28 to 30 are results according to the number of reuse of the reaction mother liquor, i.e., a chlorine source and water, recovered in Example 1, and it was confirmed that the yield per cycle of ADCA was the same regardless of the number of reuse.

Comparative Example 1 is a result obtained using urea instead of HDCA, and it was confirmed that no reaction occurred at all.

### Examples 31 to 35

An azo compound was prepared in the same manner as in Example 1, except in Table 2 below.

Regarding the quality of the ADCA obtained described in Table 2 below, the meanings of the following indications are as follows.

[symbol] : a decomposition temperature of about 207±0.5° C. (expression of an appropriate decomposition temperature),

∘: a decomposition temperature of higher than about 207.5° C. to 209.5° C. or below (slightly higher than the appropriate decomposition temperature),

Δ: a decomposition temperature of higher than about 209.5° C. (a decrease of foaming ratio performance due to delayed decomposition), and

X: a decomposition temperature of lower than about 206.5° C. (a decreased quality of a foaming body due to premature foaming)

TABLE 2

| | | | | | | | | | | Amount of | |
|---|---|---|---|---|---|---|---|---|---|---|---|
| | | | | | | | | | | Electric | |
| | | MₐX_{b} | Solvent | HDCA | | | Temper- | | | Power per 1 | |
| | | Content | Content | Content | Current | Time | ature | | Yield | g of ADCA | ADCA |
| | MₐX_{b} | (wt %) | (wt %) | (wt %) | (A) | (h) | (° C.) | pH | (%) | (W/g) | Quality |
| Example 31 | HCl | 6 | 68.99 | 25 | 10 | 5 | 40 | 1 | 94 | 1.56 | X |
| | HBr | 0.01 | | | | | | | | | |
| Example 32 | HCl | 6 | 68.9 | 25 | 10 | 5 | 40 | 1 | 95 | 1.43 | ⊚ |
| | HBr | 0.1 | | | | | | | | | |
| Example 33 | HCl | 6 | 67 | 25 | 10 | 5 | 40 | 1 | 96 | 1.42 | ⊚ |
| | HBr | 2 | | | | | | | | | |
| Example 34 | HCl | 6 | 66 | 25 | 10 | 5 | 40 | 1 | 92 | 1.4 | ◯ |
| | HBr | 3 | | | | | | | | | |
| Example 35 | HCl | 6 | 63 | 25 | 10 | 5 | 40 | 1 | 88 | 1.64 | ◯ |
| | HBr | 6 | | | | | | | | | |

In Table 2, in Examples 31 to 35, in which the content of HBr (i.e., a Br₂ precursor) was changed, it was confirmed that when the Br2 precursor was less than 0.05 wt %, the amount of electric power per 1 g of ADCA was increased, whereas when it exceeded 5 wt %, the yield was lowered while the amount of electric power per 1 g of ADCA was increased.

### Examples 36 to 41

An azo compound was prepared in the same manner as in Example 1 except that the control temperature of the temperature control unit was changed as shown in Table 3 below.

Regarding the quality of the ADCA obtained described in Table 3 below, the meanings of the following indications are as follows.

[symbol] : a decomposition temperature of about 207±0.5° C. (expression of an appropriate decomposition temperature),

∘: a decomposition temperature of higher than about 207.5° C. to 209.5° C. or below (slightly higher than the appropriate decomposition temperature),

Δ: a decomposition temperature of higher than about 209.5° C. (a decrease of foaming ratio performance due to delayed decomposition), and

X: a decomposition temperature of lower than about 206.5° C. (a decreased quality of a foaming body due to premature foaming)

TABLE 3

| | | | | | | | | | Amount of | |
|---|---|---|---|---|---|---|---|---|---|---|
| | | | | | | | | | Electric | |
| | | MₐX_{b} | Solvent | HDCA | | | Temper- | | Power per 1 | |
| | | Content | Content | Content | Current | Time | ature | | g of ADCA | ADCA |
| | MₐX_{b} | (wt %) | (wt %) | (wt %) | (A) | (h) | (° C.) | pH | (W/g) | Quality |
| Example 36 | HCl | 6 | 68.9 | 25 | 10 | 5 | 15 | 1 | 1.57 | ⊚ |
| | HBr | 0.1 | | | | | | | | |
| Example 37 | HCl | 6 | 68.9 | 25 | 10 | 5 | 30 | 1 | 1.56 | ⊚ |
| | HBr | 0.1 | | | | | | | | |
| Example 38 | HCl | 6 | 68.9 | 25 | 10 | 5 | 45 | 1 | 1.55 | ⊚ |
| | HBr | 0.1 | | | | | | | | |
| Example 39 | HCl | 6 | 68.9 | 25 | 10 | 5 | 60 | 1 | 1.93 | ◯ |
| | HBr | 0.1 | | | | | | | | |
| Example 40 | HCl | 6 | 68.9 | 25 | 10 | 5 | 75 | 1 | 2.39 | ◯ |
| | HBr | 0.1 | | | | | | | | |
| Example 41 | HCl | 6 | 68.9 | 25 | 10 | 5 | 85 | 1 | 7.12 | ◯ |
| | HBr | 0.1 | | | | | | | | |

In Table 3, in Examples 36 to 41, in which the control temperature of the temperature control unit was changed, it was confirmed that the amount of electric power per 1 g of ADCA significantly increased when the adjusted reaction temperature exceeded 80° C.

### Examples 42 and 43

An azo compound was prepared in the same manner as in Example 1, but with changes as shown in Table 4 below.

Regarding the quality of the ADCA obtained described in Table 4 below, the meanings of the following indications are as follows.

[symbol] : a decomposition temperature of about 207±0.5° C. (expression of an appropriate decomposition temperature),

∘: a decomposition temperature of higher than about 207.5° C. to 209.5° C. or below (slightly higher than the appropriate decomposition temperature),

Δ: a decomposition temperature of higher than about 209.5° C. (a decrease of foaming ratio performance due to delayed decomposition), and

X: a decomposition temperature of lower than about 206.5° C. (a decreased quality of a foaming body due to premature foaming)

TABLE 4

| | | | MₐX_{b} | | | | | | | | Electric | |
|---|---|---|---|---|---|---|---|---|---|---|---|---|
| | | MₐX_{b} | Total | Solvent | HDCA | | | Temper- | Relational | | Power per 1 | |
| | | Content | Content | Content | Content | Current | Time | ature | Equation | | g of ADCA | |
| | MₐX_{b} | (wt %) | (wt %, α) | (wt %) | (wt %) | (A) | (h) | (° C.) | (1) (α²/β)^{1/2} | pH | (W/g) | ADCA |
| Example 42 | HCl | 0.75 | 0.775 | 74.225 | 25 | 10 | 5 | 85 | 0.0841 | 1 | 7.53 | |
| | HBr | 0.025 | | | | | | | | | | |
| Example 43 | HCl | 1.75 | 1.775 | 73.225 | 25 | 10 | 5 | 75 | 0.205 | 1 | 2.71 | ◯ |
| | HBr | 0.025 | | | | | | | | | | |
| Example 44 | HCl | 2.5 | 2.575 | 72.425 | 25 | 10 | 5 | 50 | 0.364 | 1 | 1.56 | [symbol] |
| | HBr | 0.075 | | | | | | | | | | |
| Example 45 | HCl | 9 | 11.5 | 63.5 | 25 | 10 | 5 | 25 | 2.3 | 1 | 1.57 | [symbol] |
| | HBr | 2.5 | | | | | | | | | | |
| Example 46 | HCl | 16.25 | 20 | 55 | 25 | 10 | 5 | 13 | 5.547 | 1 | 1.57 | [symbol] |
| | HBr | 3.75 | | | | | | | | | | |
| Example 47 | HCl | 20 | 27.5 | 47.5 | 25 | 10 | 5 | 12 | 7.939 | 1 | 1.89 | ◯ |
| | HBr | 7.5 | | | | | | | | | | |
| Example 48 | HCl | 22.5 | 31.25 | 43.75 | 25 | 10 | 5 | 7 | 11.811 | 1 | 3.15 | X |
| | HBr | 8.75 | | | | | | | | | | |

In Table 4, when the value of Relational Equation (1) is less than 0.1, it was confirmed that the amount of electric power per lg of ADCA was significantly increased and the quality was deteriorated.

### Comparative Example 2

An azo compound was prepared in the same manner as in Example 1, except that chlorine gas was directly introduced without electrolysis.

TABLE 5

| | | | | | | | | | | | | Content of |
|---|---|---|---|---|---|---|---|---|---|---|---|---|
| | | | | | | | | | | Content | | Wastewater |
| | | MₐX_{b} | Solvent | HDCA | | | Temper- | | | of ADCA | | Production |
| | | Content | Content | Content | Current | Time | ature | | Yield | Acquired | ADCA | (g of HCl per |
| | MₐX_{b} | (wt %) | (wt %) | (wt %) | (A) | (h) | (° C.) | pH | (%) | (g) | Quality | kg ADCA) |
| Example 33 | HCl | 6 | 67 | 25 | 10 | 5 | 40 | 1 | 96 | 23.6 | [symbol] | None |
| | HBr | 2 | | | | | | | | | | |
| Comparative | Cl₂ | 15 | 75 | 25 | 10 | — | 40 | 1 | 94 | 23.1 | ◯ | 627.8 g |
| Example 2 | | | | | | | | | | | | |

In Table 5, in Comparative Example 2, chlorine gas was directly introduceed without performing an electrolysis reaction, and ADCA was obtained in a high yield of 94%, but it was confirmed that there were problems in that a large amount of chlorine source had to be continuously introduceed, and 627.8 g of HCl was produced per 1 kg of ADCA but the HCl could not be reused, thus requiring wastewater treatment, and in that the HCl had to be neutralized using a large amount of an alkali compound for the wastewater treatment.

As described above, according to the embodiments of the present invention, it is possible to implement a device for producing an azo compound, in which it is not necessary to continuously introduce a chlorine source, etc. through a recycling process because a predetermined halogen compound (MₐX_{b}) is used, it can significantly reduce the burden of treatment of wastewater and by-products, and realize a high conversion rate and a high yield. Additionally, even if the electrolysis method is used, it is unnecessary to use a separator, and it is possible to implement a device for producing an azo compound capable of reducing electric power consumption compared to the convenitonal technology. Accordingly, the manufacturing process and process management can be easier, manufacturing cost can be reduced, and productivity can be improved.

In the present specification, preferred embodiments of the present invention have been disclosed. Although specific terms are used, these are only used in a general sense to easily describe the technical contents of the present invention and help the understanding of the present invention, but it is not meant to limit the scope of the present invention. It will be apparent to those of ordinary skill in the art to which the present invention pertains that other modifications based on the technical spirit of the present invention can be implemented, in additional to the embodiments disclosed herein. For example, those of ordinary skill in the art would be able to understand that the method for producing an azo compound according to the embodiments described with reference to FIGS. 2 to 5 and the reaction systsem that can be used for the same (i.e., a device for preparing an azo compound) can be variously modified. Therefore, the scope of the invention should not be determined by the described embodiments, but should be determined by the technical ideas described in the claims.

### REFERENCE NUMERALS

3A: reaction solution introduction unit

3B: hydrazo compound introduction unit

6: discharge unit

7: dehydration unit

8: dehydration mother liquor storage tank

9: recycling unit

10: vessel

11: negative electrode

12: positive electrode

13: separator

14: negative electrode compartment

15: positive electrode compartment

16: stirrer

20, 25: reaction tanks

35a, 35b: connecting pipes

45: pump

46: reaction solution transfer pump

55: electrode chamber

60A, 65A: negative electrodes

60B, 65B: positive electrodes

70, 75: stirrers

85: gas treatment unit

17, 100: solutions

S10: first step

S20: second step

S30: third step

S40: fourth step

1. A method for preparing azo compound, comprising:
a first step for producing an X_{b} molecule by electrolyzing, in a reaction system, a first solution including a hydrazo compound and at least one type of MₐX_{b};
a second step for oxidizing the hydrazo compound with the generated X_{b} molecule to obtain a second solution including an azo compound, MₐX_{b}, and HX;
a third step for discharging the second solution outside the reaction system, and separating therefrom a third solution including MₐX_{b} and HX to obtain a solid azo compound; and
a fourth step for introducing the third solution and an additional hydrazo compound equivalent to the hydrazo compound into the reaction system, and electrolyzing a fourth solution including the additional hydrazo compound, MₐX_{b}, and HX to produce an X_{b} molecule,
wherein the fourth step, the second step, and the third step are collectively defined as a single cycle, and the cycle is performed repeatedly, and
wherein:
the X is a halogen element;
the M is at least one selected from the group consisting of hydrogen, Li, Na, K, Mg, Ca, Mn, Fe, Ni, Cu, Ag, Zn, Sn, Zr, and Ti, or at least one selected from the group consisting of a primary ammonium ion, a secondary ammonium ion, and a tertiary ammonium ion;
the H is hydrogen; and
a and b are each independently any one integer from 1 to 4.

2. The method of claim 1, wherein the content of the at least one type of MₐX_{b} initially introduced into the reaction system in the first step is 1 wt % to 30 wt % based on the total weight of the first solution.

3. The method of claim 1, wherein the MₐX_{b} comprises at least one of a Cl₂ precursor and a Br₂ precursor.

4. The method of claim 3, wherein the MₐX_{b} comprises a Cl₂ precursor and the contentcontentof the Cl₂ precursor is 3 wt % to 15 wt % based on the total weight of the first solution.

5. The method of claim 3,
wherein the MₐX_{b} comprises both a Cl₂ precursor and a Br₂ precursor, and
wherein the content of the Cl₂ precursor is 3 w % to 15 w % based on the total weight of the first solution, and the content of the Br2 precursor is 0.05 wt % to 5 wt % based on the total weight of the first solution.

6. The method of claim 1, wherein the method for producing an azo compound satisfies the following Relational Equation (1),
$$0.1 \leq \sqrt{\frac{{α}^{2}}{β}} \leq 9.5 \qquad [ Relational Equation ( 1 ) ]$$
wherein in Relational Equation (1) above, α is the content (wt %) of MₐX_{b} based on the total weight of the first solution, and β is the reaction temperature (° C.) of the method for producing an azo compound.

7. The method of claim 1, wherein the method for producing an azo compound satisfies the following Relational Equation (1-1),
$$0.33 \leq \sqrt{\frac{{α}^{2}}{β}} \leq 6.35 \qquad [ Relational Equation ( 1 - 1 ) ]$$
wherein in Relational Equation (1-1) above, α is the content (w %) of MₐX_{b} based on the total weight of the first solution, and β is the reaction temperature (° C.) of the method for producing an azo compound.

8. The method of claim 1, wherein the concentration of HX is maintained uniformly in the first to third solutions from the starting time of the first step to the ending time of the third step.

9. The method of claim 1, wherein the reaction system comprises a solution of any one of the first to fourth steps; a positive electrode immersed in the solution; and a negative electrode immersed in the solution; and wherein the solution around the positive electrode and the negative electrode is acidic.

10. The method of claim 9, wherein the negative electrode is in direct contact with any one or more of the hydrazo compound and the azo compound.

11. The method of claim 1, wherein the hydrazo compound is hydrazodicarbonamide (HDCA) and the azo compound is azodicarbonamide (ADCA).

12. The method of claim 1, wherein the azo compound is present in a slurry state before separating the third solution in the third step.

13. A method for preparing azo compound, comprising:
a first step for producing an X_{b} molecule by electrolyzing a first solution including a hydrazo compound and at least one type of MₐX_{b}, in a reaction system;
a second step for oxidizing the hydrazo compound with the generated X_{b} molecule to obtain a second solution including an azo compound, MₐX_{b}, and HX;
a third step for discharging the second solution outside the reaction system, and separating therefrom a third solution including MₐX_{b} and HX to obtain a solid azo compound;
wherein the pH of the first solution to the third solution in the first step to third step reaction systems is uniform,
wherein:
the X is a halogen element;
the M is at least one selected from the group consisting of hydrogen, Li, Na, K, Mg, Ca, Mn, Fe, Ni, Cu, Ag, Zn, Sn, Zr, and Ti, or at least one selected from the group consisting of a primary ammonium ion, a secondary ammonium ion, and a tertiary ammonium ion;
the H is hydrogen; and
a and b are each independently any one integer from 1 to 4.

14. The method of claim 13, wherein the content of the at least one type of MₐX_{b} initially introduced into the reaction system in the first step is 1 wt % to 30 wt % based on the total weight of the first solution.

15. The method of claim 13, wherein the MₐX_{b} comprises at least one of a Cl₂ precursor and a Br₂ precursor.

16. The method of claim 15, wherein the MₐX_{b} comprises a Cl₂ precursor and the content of the Cl₂ precursor is 3 wt % to 15 wt % based on the total weight of the first solution.

17. The method of claim 15,
wherein the MₐX_{b} comprises both a Cl₂ precursor and a Br₂ precursor, and
wherein the content of the Cl₂ precursor is 3 w % to 15 w % based on the total weight of the first solution, and the content of the Br₂ precursor is 0.05 wt % to 5 wt % based on the total weight of the first solution.

18. The method of claim 13, wherein the concentration of HX is maintained uniformly in the first to third solutions from the starting time of the first step to the ending time of the third step.

19. The method of claim 13, wherein the method further comprises a fourth step for introducing an additional hydrazo compound equivalent to the hydrazo compound and the third solution into the reaction system, and electrolyzing a fourth solution including the additional hydrazo compound, MₐX_{b}, and HX to produce an X_{b} molecule.

20. The method of claim 19, wherein the fourth step, the second step, and the third step are collectively defined as a single cycle, and the cycle is performed repeatedly.

21. The method of claim 13,
wherein the reaction system comprises a solution of any one of the first to fourth steps; a positive electrode immersed in the solution; and a negative electrode immersed in the solution; and
wherein the solution around the positive electrode and the negative electrode is acidic.

22. The method of claim 21, wherein the negative electrode is in direct contact with any one or more of the hydrazo compound and the azo compound.

23. The method of claim 13, wherein the hydrazo compound is hydrazodicarbonamide (HDCA) and the azo compound is azodicarbonamide (ADCA).

24. The method of claim 13, wherein the azo compound is present in a slurry state before separating the third solution in the third step.
